(19) United States
(12) Patent Application Publication
(10) Pub. No.: US 2024/0065693 A1
(43) Pub. Date: Feb. 29, 2024

(54) DEVICES, SYSTEMS, AND METHODS FOR CONNECTING ENDS OF ANATOMICAL STRUCTURES

(71) Applicant: BOSTON SCIENTIFIC SCIMED, INC., Maple Grove, MN (US)

(72) Inventors: Yeison Calvo, San Ramón (CR); Gonzalo Jose Saenz Villalobos, Alajuela (CR); Gian Franco Loo Fuchs, San Jose (CR)

(73) Assignee: BOSTON SCIENTIFIC SCIMED, INC., Maple Grove, MN (US)

(21) Appl. No.: 18/238,642

(22) Filed: Aug. 28, 2023

Related U.S. Application Data
(60) Provisional application No. 63/401,778, filed on Aug. 29, 2022.

(51) Int. Cl. A61B 17/11; A61F 2/04

(52) U.S. Cl. CPC A61B 17/11; A61B 17/1114; A61F 2/04; A61B 2017/1132; A61F 2002/045

(57) ABSTRACT

An implantable medical device system for coupling separate anatomical structures, such as to form an anastomosis therebetween. The system includes first and second implantable medical devices configured to be implanted with respect to anatomical structures to be coupled, and also configured to be coupled together. The implantable medical devices may be coupled together with a separate connector. The separate connector may be a stent or an elongated tether-like element. Additionally or alternatively, the implantable medical devices may be formed with interengaging elements which couple the implantable medical devices together, such as without the need for a separate connector. The implantable medical devices may be spaced apart when coupled together, with a connector bridging the gap therebetween, or may be in abutting contact with each other.

### CROSS-REFERENCE TO RELATED APPLICATIONS

This application claims the benefit of priority under 35 U.S.C. § 119 to U.S. Provisional Application No. 63/401,778, filed Aug. 29, 2022, the entire disclosure of which is hereby incorporated by reference herein for all purposes.

### FIELD

The present disclosure relates generally to the field of implantable medical devices. In particular, the present disclosure relates to implantable medical devices, systems, and methods for joining anatomical structures, such as ends of body lumens, such as to form an anastomosis. More particularly, the devices, systems, and methods of the present disclosure may be used transluminally without requiring open surgical procedures such as those requiring incisions to access the internal anatomical structures to be joined.

### BACKGROUND

Various tissue dissection or resection procedures are known for cutting and/or removing anatomical tissue from a target area within the body, such as to treat various pathologies. Tissue dissection procedures typically involve cutting tissue, and tissue resection procedures typically involve cutting and removing tissue and/or a part of an anatomical structure (e.g., an organ, tissue wall, body lumen, etc.). Full-thickness resection of tissue, through multiple layers of a tissue wall, may be performed, such as to remove harmful growths or lesions. Dissections and resections of body lumens (with generally tubular tissue walls), such as tissue of a gastrointestinal tract, may be particularly challenging because the integrity of the lumen may be important to prevent materials from passing through the lumen wall and out of the lumen rather than through and along the longitudinal extent of the lumen.

Open surgery (involving cutting open the patient's body to gain access into the patient's body and internal anatomical structures) or laparoscopy (typically involving smaller incisions to gain access into the patient's body and internal anatomical structures) are the most common methods for performing tissue resection. Such methods involve longer recovery times than endoscopic methods which do not involve incisions, but which, instead, typically access the internal anatomy through a natural orifice and advancing equipment, tools, devices, etc., transluminally to the target area. However, full-thickness endoscopic resections of target tissue (e.g., tissue comprising the deleterious growth or lesion) present various other challenges, such as with visualizing adjacent anatomy during the procedure.

Accordingly, there remains an ongoing need for endoscopic devices, systems, and methods for dissecting and/or resecting tissue, such as dissecting and/or resecting body lumens. There remains an associated ongoing need for endoscopic devices, systems, and methods for creating an anastomosis between resected sections of a body lumen.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an implantable medical device system configured to form an anastomosis across cut ends of a body lumen includes a first tubular implantable medical device, a second tubular implantable medical device, and a connector configured to couple the first tubular implantable medical device and the second tubular implantable medical device together. In some aspects, the first tubular implantable medical device and the second tubular implantable medical device include tissue-engagement features configured to facilitate coupling of the first and second tubular implantable medical devices with respect to a body lumen, and a support configured to facilitate coupling of the first and second tubular implantable devices together.

In some embodiments, the tissue-engagement features are provided at a tissue-engaging region of each of the first and second implantable medical devices; and the support includes a support at a device-engaging region of each of the first and second implantable medical devices.

In some embodiments, the tissue-engagement features comprise apertures.

In some embodiments, the tissue-engagement features are configured to allow at least one of a tissue connector or tissue to extend therethrough to anchor the first and second implantable medical devices with respect to the body lumen.

In some embodiments, the connector is a tubular connector formed separately from the first and second implantable medical devices, and has a first end engageable with the support of the first implantable medical device and a second end engageable with the support of the second implantable medical device to couple the first and second implantable medical devices together. In some embodiments, the tubular connector couples the first and second implantable medical devices together with supports of the first and second implantable medical device spaced apart from each other. In some embodiments, the tubular connector is coated to prevent passage of materials through the wall thereof. In some embodiments, the tubular connector couples the first and second implantable medical devices together with supports of the first and second implantable medical device abuttingly engaging each other.

In some embodiments, the connector is an elongated tether-like connector formed separately from the first and second implantable medical devices and configured to be secured to the supports of the first and second implantable medical devices to couple the first and second implantable medical devices together in abutting engagement.

In some embodiments, the connector comprises interengaging features on the supports of the first and second implantable medical device configured to interengage each other to couple the first and second implantable medical devices together.

In some embodiments, the system further comprises a tether coupling the first and second implantable medical devices together after the body lumen to which the first and second implantable medical devices are coupled has been cut between the first and second implantable medical devices.

In accordance with various principles of the present disclosure, an implantable medical device system includes a first implantable medical device; a second implantable medical device; and a connector configured to couple the first implantable medical device and the second implantable medical device together. In some aspects, the first implantable medical device and the second implantable medical device each include tissue-engagement features configured to facilitate coupling of the first and second implantable medical devices with tissue; and a support configured to facilitate engagement of the first and second implantable devices to couple the first and second implantable medical devices together. In some aspects, the connector establishes an anastomosis through the first implantable medical device and the second implantable medical device preventing flow of materials in a direction through walls of the first and second implantable medical devices.

In some embodiments, the tissue-engagement features comprise apertures configured to allow at least one of a tissue connector or tissue to extend therethrough.

In some embodiments, the connector is a tubular connector formed separately from the first and second implantable medical devices, and has a first end engageable with the support of the first implantable medical device and a second end engageable with the support of the second implantable medical device to couple the first and second implantable medical devices together. In some embodiments, the tubular connector is a stent having a first end engageable with the support of the first implantable medical device and a second end engageable with the support of the second implantable medical device.

In accordance with various principles of the present disclosure, a method of forming an anastomosis after a colostomy includes implanting a first implantable medical device within a colon at a location distal to target tissue to be excised; implanting a second implantable medical device within a colon at location proximal to target tissue to be excised; excising the target tissue; and joining device-engaging regions of the first and second implantable medical devices with a connector.

In some aspects, implanting a first implantable medical device and implanting a second implantable medical device comprise extending at least one of tissue of the colon or a tissue connector through tissue-engagement features in each of the first implantable medical device and the second implantable medical device to anchor the first and second implantable medical devices with respect to the colon.

In some aspects, joining device-engaging regions of the first and second implantable medical devices comprises extending a tubular stent between the first and second implantable medical devices to form an anastomosis therebetween.

In some aspects, joining device-engaging regions of the first and second implantable medical devices comprises bringing the first and second devices into abutting engagement and coupling the first and second implantable medical devices together in abutting engagement to form an anastomosis through the first and second implantable medical devices.

In some aspects, the method further includes coupling a portion of the colon distal to the first implantable medical device with a portion of the colon proximal to the second implantable medical device with a tether before joining the first and second implantable medical devices together.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the various figures, identical or nearly identical or equivalent elements are typically represented by the same reference characters, and similar elements are typically designated with similar reference numbers followed by ‘, or ‘′, with redundant description omitted for the sake of brevity. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an implantable device formed in accordance with various aspects of the present disclosure and positioned in a schematic representation of a gastrointestinal environment.

FIG. 2 illustrates a perspective view of an example of an implantable medical device of an implantable medical device system formed in accordance with various principles of the present disclosure.

FIG. 3 is a cross-sectional view along line II-II of FIG. 2.

FIGS. 4A, 4B, 4C, and 4D illustrate views along detail 4 in FIG. 3.

FIG. 5 illustrates a cross-sectional view of an implantable medical device formed in accordance with various principles of the present disclosure deployed in a schematic representation of a body lumen.

FIG. 6A illustrates a cross-sectional view of an implantable medical device such as illustrated in FIG. 5 being implanted with respect to a schematic representation of body tissue with an example of an embodiment of a tissue connector.

FIG. 6B illustrates a cross-sectional view of an implantable medical device such as illustrated in FIG. 5 implanted with respect to a schematic representation of body tissue with an example of an embodiment of a tissue connector.

FIG. 6C illustrates a cross-sectional view of an implantable medical device such as illustrated in FIG. 5 being implanted with respect to a schematic representation of body tissue with an example of an embodiment of a tissue connector.

FIG. 7A illustrates a schematic representation of an example of an embodiment of a tissue connector within an example of an embodiment of a delivery sheath.

FIG. 7B illustrates a schematic representation of an example of an embodiment of a tissue connector such as illustrated in FIG. 7A being deployed from a delivery sheath such as illustrated in FIG. 7A.

FIG. 7C illustrates a schematic representation of an example of an embodiment of a tissue connector such as illustrated in FIG. 7A deployed from a delivery sheath such as illustrated in FIG. 7A.

FIG. 8 illustrates a cross-sectional view of an implantable medical device system, including first and second implantable medical devices such as illustrated in FIG. 5, implanted with respect to a schematic representation of a body lumen.

FIG. 9 illustrates a view similar to that of FIG. 8, with a tether extending within the body lumen and across the illustrated example of an embodiment of an implantable medical device system.

FIG. 10 illustrates a view similar to that of FIG. 9, with an endoscope delivering a cutting tool, and illustrating the cutting tool cutting the schematically illustrated body lumen wall.

FIG. 11 illustrates a view similar to that of FIG. 10, with the schematic representation of a body lumen cut along and proximal to one of the implantable medical devices of the illustrated example of an embodiment of an implantable medical device system, and illustrating the cutting tool cutting the schematically illustrated body lumen wall distal to the other of the implantable medical devices.

FIG. 12 illustrates a view similar to that of FIG. 11, with the schematic representation of a body lumen cut along and distal to the other of the implantable medical devices of the illustrated example of an embodiment of an implantable medical device system, and with a tool grasping the schematically illustrated resected tissue between the implantable medical devices of the illustrated example of an embodiment of an implantable medical device system.

FIG. 13 illustrates a view similar to that of FIG. 12, but with the tool retracting the schematically illustrated resected tissue proximally.

FIG. 14 illustrates a view similar to that of FIG. 13, but with the schematically illustrated resected tissue removed.

FIG. 15A illustrates a view similar to that of FIG. 14, but with an example of an embodiment of a connector being deployed to couple the implantable medical devices of the illustrated example of an embodiment of an implantable medical device system.

FIG. 15B illustrates a view similar to that of FIG. 15A, but with the connector deployed and coupling the implantable medical devices of the illustrated example of an embodiment of an implantable medical device system.

FIG. 16 illustrates another example of an embodiment of a connector coupling the implantable medical devices of the illustrated example of an embodiment of an implantable medical device system.

FIG. 17 illustrates another example of an embodiment of a connector coupling the implantable medical devices of the illustrated example of an embodiment of an implantable medical device system.

FIG. 18 illustrates another example of an embodiment of a connector coupling the implantable medical devices of the illustrated example of an embodiment of an implantable medical device system.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site. As such, reference to “at” a target tissue site is intended to include tissue at and about the vicinity of (e.g., along, adjacent, etc.) the target tissue, and is not limited to just target tissue. References to a target tissue site, deployment site, target area, etc., may be made interchangeably herein without intent to limit.

In accordance with various principles of the present disclosure, an implantable medical device system includes a first implantable medical device configured to be coupled to a body lumen and a second implantable medical device configured to be coupled to a body lumen. Reference is made to a body lumen for the sake of convenience and without intent to limit, and such term is to be understood herein as referring to an anatomical structure (e.g., tissue wall) forming a passage therethrough. Moreover, it will be appreciated that terms such as deploy, implant, anchor, attach, associate, secure, couple, join, engage, etc., (and various grammatical forms thereof), may be used interchangeably herein without intent to limit. The first and second implantable medical devices of the implantable medical device system are deployed within the body lumen and/or around the body lumen. However, other relative positions of the devices and body lumen are within the scope and spirit of the present disclosure as may be appreciated by one of ordinary skill in the art.

In some embodiments, the implantable medical devices are implanted while the body lumen intact. For instance, one implantable medical device of the implantable medical device system may be deployed and implanted distal to the target area of tissue to be treated and/or excised, and another implantable medical device of the implantable medical device system may be deployed and implanted proximal to the target area of tissue to be treated and/or excised. The order of deployment is not critical, though it may be simpler to deploy the distal of the implantable medical devices before the proximal of the implantable medical device. If implanted together in an intact body lumen, the implantable medical devices may be implanted spaced apart from each other, and the body lumen may then be cut between the respective deployment sites of the first implantable medical device and the second implantable medical device. Tissue of the body lumen, such as between the deployment sites of the first and second implantable medical devices, may then be removed (e.g., if diseased, damaged, malformed, etc.). The separated cut ends of the body lumen are then joined together via the implantable medical devices and system of the present disclosure. Use of an implantable medical device system formed in accordance with various principles of the present disclosure may thereby reestablish flow of materials through the disrupted body passage by facilitating rejoining of the ends of the body lumen, such as to form an anastomosis. It will be appreciated that in some embodiments, a system and associated devices as disclosed herein may be coupled with already separate ends of tubular structures in a patient's body. Reference may be made herein interchangeably to terms such as cut, dissect, resect, excise, incise, remove, etc., and other grammatical forms thereto, without intent to limit.

The use of separately formed implantable medical devices in some embodiments may be beneficial, such as to allow independent delivery of the devices, independent deployment of the devices, independent adjustability of the devices and the overall system, increased adjustability of the overall system, reduction in material needed to span across an area to be joined by the devices, etc., as will become apparent in light of the following disclosure.

In accordance with various principles of the present disclosure, at least a portion of the implantable medical device system, such as at least a portion of at least one of, or both of, the implantable medical devices has an attachment feature configured to facilitate attachment to the tissue wall of the deployment site (e.g., a body lumen wall). For instance, at least a portion of the implantable medical device system is configured to be directly engaged with tissue at the deployment site (e.g., having a portion clipped, anchored, penetrating, or otherwise engaging tissue at the deployment site to maintain the implantable medical device system positioned with respect to the deployment site), and/or to allow a separate coupler (e.g., tether, wire, suture, string, staple, bonding agent, etc.) to be engaged with both the implantable medical device system and tissue at the deployment site, and/or to allow tissue ingrowth with respect to the implantable medical device system.

Moreover, in accordance with various principles of the present disclosure, the first implantable medical device and the second implantable medical device of an implantable medical device system are configured to be coupled with each other along at least a portion thereof. For instance, in some embodiments, each of the first implantable medical device and the second implantable medical device is configured to be engaged with a connector which extends between and couples the first and second implantable medical devices. In some embodiments, the implantable medical devices engage each other, either with a separately-formed connector therebetween, or directly (without the use of a connector, and/or with a connector formed integrally with and as a part of one or both of the implantable medical devices). For instance, in some embodiments, the first and second implantable medical devices are configured to directly engage each other generally to abut each other and to be coupled to connect body lumens in which each implantable medical device is respectively deployed.

Various embodiments of implantable medical devices, an implantable medical device system, and methods associated therewith will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

It will be appreciated that common features in the accompanying drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). For the sake of simplicity, certain features in one embodiment may be used across different embodiments and are not necessarily individually labeled when appearing in different embodiments.

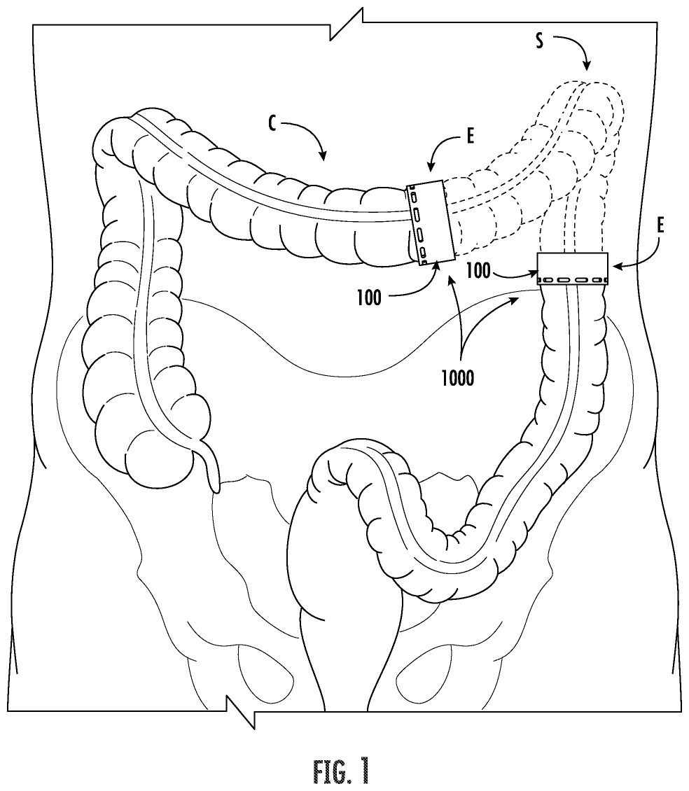

Turning now to the drawings, an example of human anatomical structure at which an anastomosis may be formed in accordance with various principles of the present disclosure is illustrated schematically in FIG. 1. An implantable medical device system 1000 formed in accordance with various principles of the present disclosure is illustrated as deployed at the illustrated anatomical site and forming an anastomosis between ends of body passages at the anatomical site. The deployment site illustrated in FIG. 1 is a gastrointestinal system, and the body passage is a colon. More particularly, the implantable medical device system 1000 is illustrated as implanted in a section of a human colon C, schematically illustrated showing cut ends E at either side of a colon section S (illustrated in phantom) to be removed or resected. However, other deployment sites and positions of an implantable medical device system 1000 are contemplated and are within the scope and spirit of the present disclosure.

The implantable medical device system 1000 illustrated in FIG. 1 includes a first implantable medical device 100 and a second implantable medical device 100 implanted with respect to a body lumen, e.g., the colon C, at either side of a section S of the body lumen to be removed. The same reference numeral, 100, is used to indicate both implantable medical devices 100 since the devices may be substantially identical. However, such use of a common reference numeral should not be taken as limiting the first and second implantable medical devices 100 to being identical. In some embodiments, the implantable medical devices 100 are not identical, yet may be matingly configured to form a set, such as an interengaging set, as will be described in further detail below. For the sake of convenience and without intent to limit, only one implantable medical device is described in detail, one of ordinary skill in the art appreciating that the description of one implantable medical device 100 of an implantable medical device system 1000 formed in accordance with various principles of the present disclosure is generally applicable to the other of the implantable medical devices of the implantable medical device system 1000.

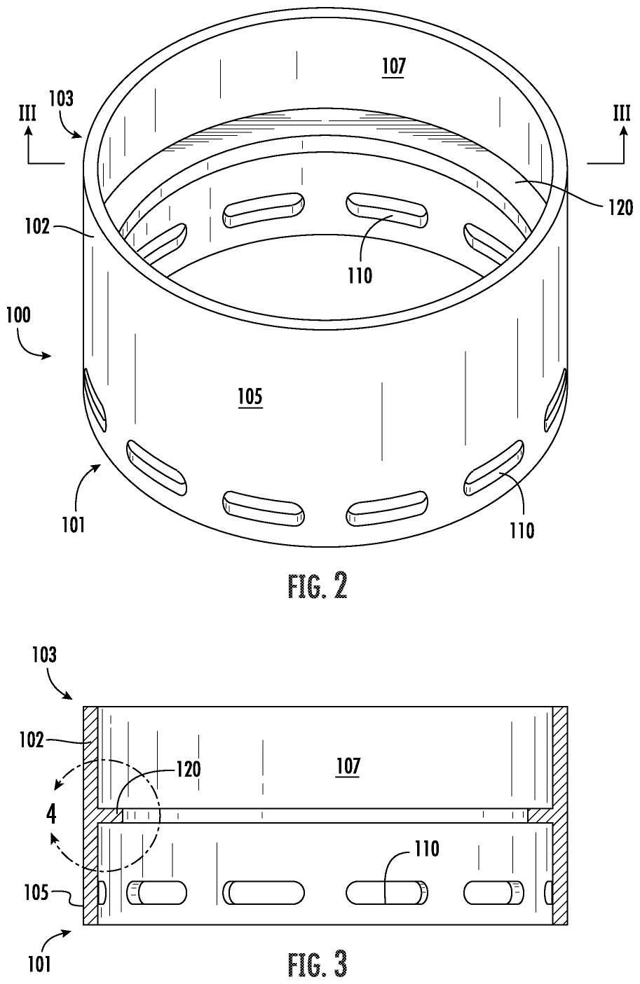

An example of an embodiment of an implantable medical device 100 of an implantable medical device system 1000 formed in accordance with various principles of the present disclosure is illustrated in further detail in FIG. 2. To facilitate transluminal (e.g., endoscopic or transcatheteral) delivery, the implantable medical device 100 may have a wall 102 capable of shifting (the wall 102 and/or the implantable medical device 100) between a delivery configuration and a deployed configuration. In the delivery configuration, at least the wall 102 is collapsed, contracted, compressed, constrained, restrained, etc., in a generally compact configuration to fit within a tubular delivery element capable of navigating within a body (such as known to those of ordinary skill in the art), such as entering the body through natural orifices (i.e., without requiring incisions to access internal anatomical structures) and travelling through tortuous internal cavities or passages to a deployment site. In the deployed configuration, at least the wall 102 is in an unconstrained, unrestrained, expanded, etc., configuration capable of engaging the wall of an anatomical structure. In embodiments configured to be coupled with respect to a body lumen, the implantable medical device 100 has a generally cylindrical or tubular wall 102.

The implantable medical device 100 may be formed as an expandable scaffold or stent formed of members (e.g., struts, wires, strands, filaments, etc.) which are braided, interengaged, intertwined, interwoven, knitted, knotted, looped (e.g., bobbinet-style), weaved, woven, wrapped, or the like to form a tubular structure. Additionally or alternatively, the implantable medical device 100 may be formed by cutting (e.g., by laser-cutting) a tubular structure (e.g., an, optionally monolithic, cylindrical tubular member) into an expandable configuration, the cuts forming members such as strut members. An implantable medical device of an implantable medical device system 1000 of the present disclosure may be a self-expanding device such as known or heretofore known to those of ordinary skill in the art. For instance, the implantable medical device may be formed of shape-memory or heat-formable material (e.g., Nitinol or Elgiloy® or shape memory polymers) so that the implantable medical device returns to a pre-shaped expanded configuration from a collapsed delivery configuration upon advancement from a delivery element and/or withdrawal of a delivery sheath which maintains the implantable medical device in a delivery configuration therein.

An example of an embodiment of an implantable medical device 100 is illustrated in further detail in FIG. 2 as having a tissue-engaging region 101 and a device-engaging region 103. The tissue-engaging region 101 may be positioned along a first end of the implantable medical device 100, and the device-engaging region 103 may be positioned along an opposite, second end of the implantable medical device 100. More particularly, the tissue-engaging region 101 may be formed along a portion of the wall 102 along a first end of the implantable medical device 100, and the device-engaging region 103 may be formed along a portion of the wall 102 along a second end of the implantable medical device 100.

In the example of an embodiment illustrated in FIG. 2, and in the cross-sectional view thereof illustrated in FIG. 3, the tissue-engaging region 101 of the implantable medical device 100 includes one or more (e.g., two, three, four, or more, depending on the circumferential extent of the wall 102 of the implantable medical device 100) tissue-engagement features 110 configured to facilitate engagement of the implantable medical device 100 with tissue at the deployment site. More particularly, in the illustrated example of an embodiment, the tissue-engagement features 110 are apertures through the wall 102 of the implantable medical device 100, extending from the outer surface 105 to the inner surface 107 of the wall 102. In some embodiments, the tissue-engagement features 110 are configured to allow tissue ingrowth therein and/or therearound. Additionally or alternatively, in some embodiments, the tissue-engagement features 110 are configured to allow insertion of a separate tissue connector therethrough to couple the implantable medical device 100 with tissue at the deployment site. The separate tissue connector may be one or more of a suture, a thread, a wire, a tether, a filament, a band, a staple, etc., or any other acceptable element known to those of ordinary skill in the art for connecting an implant with body tissue, such as described in further detail below. In some embodiments, the tissue-engagement features 110 may be configured as anchoring features facilitating direct engagement of the implantable medical device 100 with tissue. For instance, the tissue-engagement features 110 may be configured to penetrate tissue, such as an anchor, barb, etc., or otherwise to directly connect the implantable medical device 100 with tissue at the deployment site, such as in a manner known to those of ordinary skill in the art.

In the example of an embodiment illustrated in FIG. 2, and in the cross-sectional view thereof illustrated in FIG. 3, the device-engaging region 103 of the implantable medical device 100 includes a support 120, such as a ledge, ridge, projection, etc., extending from the inner surface of the wall 102 of the implantable medical device 100. The support 120 is configured to engage with the other implantable medical device 100 of the implantable medical device system 1000 to couple the implantable medical devices 100 together to form an anastomosis between ends of separate/separated body lumens. In some embodiments, the support 120 is configured to be coupled with another implantable medical device 100 with the use of a connector configured to facilitate coupling of the implantable medical devices 100 and the body lumens with which they are coupled (the body lumens being coupled such as in a manner described above or in another manner). Various examples of embodiments of connectors are described in further detail below. Additionally or alternatively, the support 120 is configured to be coupled directly with another implantable medical device 100, such as with an interengaging mating support 120 on the other implantable medical device 100, as described in further detail below.

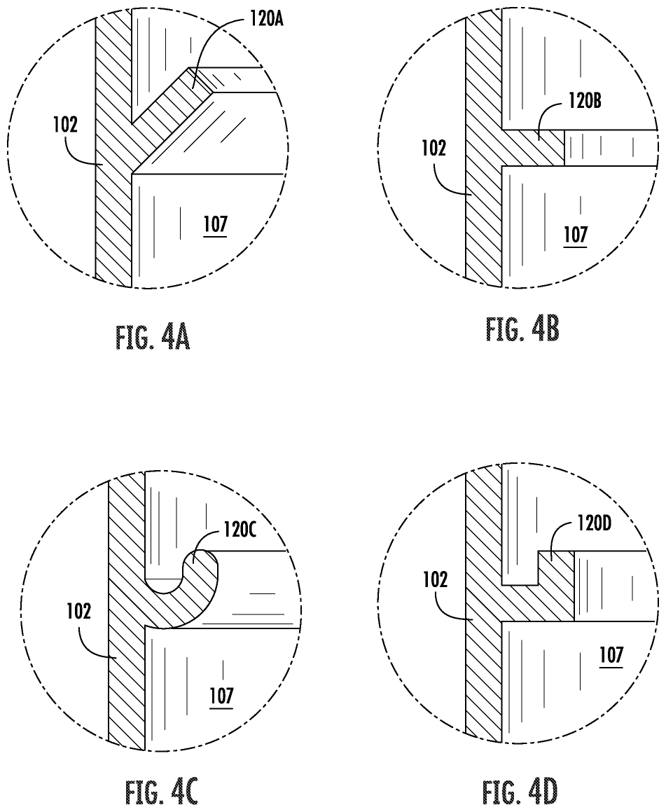

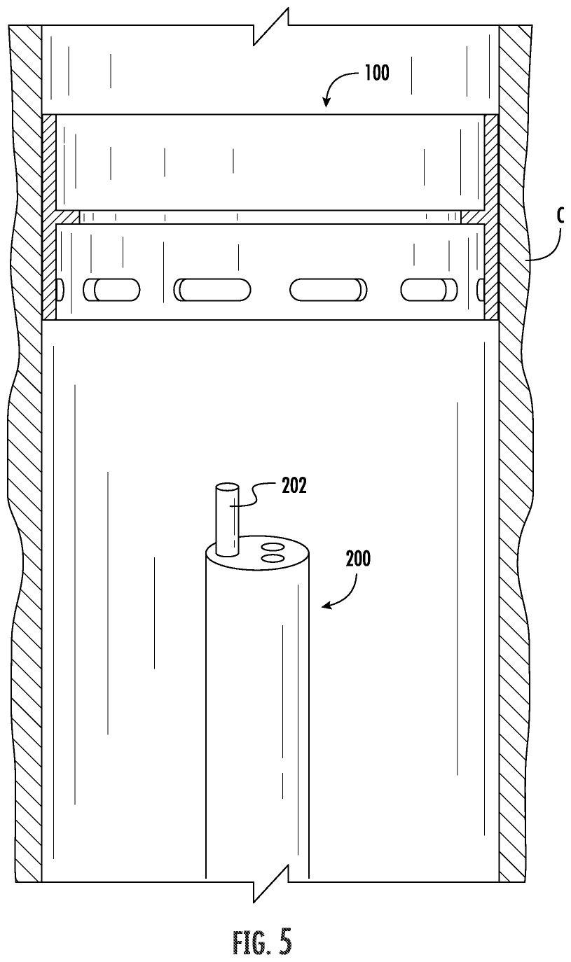

It will be appreciated that a support 120 of a device-engaging region 103 of an implantable medical device 100 formed in accordance with various principles of the present disclosure may be in any of a variety of configurations, such as to accommodate various manners of connecting two implantable medical devices 100 with the assistance of the support 120. For instance, various configurations of supports 120 are illustrated in the detail views FIGS. 4A-4D of detail 4 in FIG. 3. In particular, the support 120 may be angled with respect to the wall 102 of the implantable medical device 100 (as illustrated in FIG. 4A); or, more particularly, may extend generally perpendicularly from the inner surface 107 of the wall 102 of the implantable medical device 100 (as illustrated in FIG. 4B); and/or may be curved with respect to the inner surface 107 of the wall 102 of the implantable medical device 100 such as to form a curved hook-like configuration (as illustrated in FIG. 4C), and/or a more straight/angular hook (as illustrated in FIG. 4D). It will be appreciated that the scope of the present disclosure need not be limited to a specific configuration of a support 120 such as illustrated in FIGS. 4A-4D.

Various examples of manners of implanting implantable medical devices 100 within respective portions of body lumens, such as with tissue-engagement features 110 as described above, and joining body lumens with the assistance of the implantable medical devices 100, such as with a support 120 as described above, may be appreciated with reference to FIGS. 5-18. Implantable medical devices 100 of an implantable medical device system 1000 formed in accordance with various principles of the present disclosure may be deployed at a first site in a body lumen in any of a variety of manners known to those of ordinary skill in the art, the details of which are not critical to the present disclosure and thus not provided. However, it will be appreciated that the implantable medical device system 1000 preferably is capable of transluminal, such as endoscopic, delivery, such as within a working channel of an endoscope 200 (and optionally within a generally tubular delivery element within a working channel of the endoscope 200) as illustrated in FIG. 5. Once an implantable medical device 100 is deployed, such as in a colon C, an appropriate working instrument 202 may be delivered, such as with the endoscope 200, to anchor the implantable medical device 100 with respect to the deployment site (in the illustrated example of an embodiment, the internal wall of a colon C).

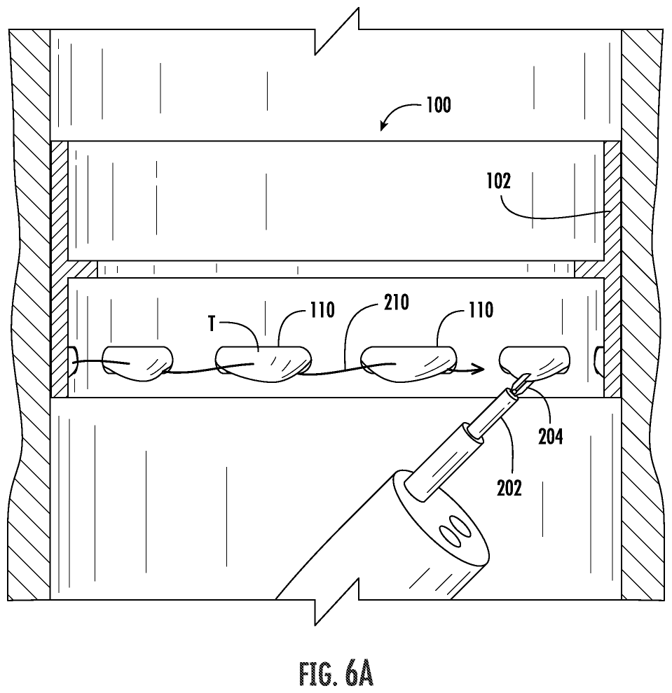

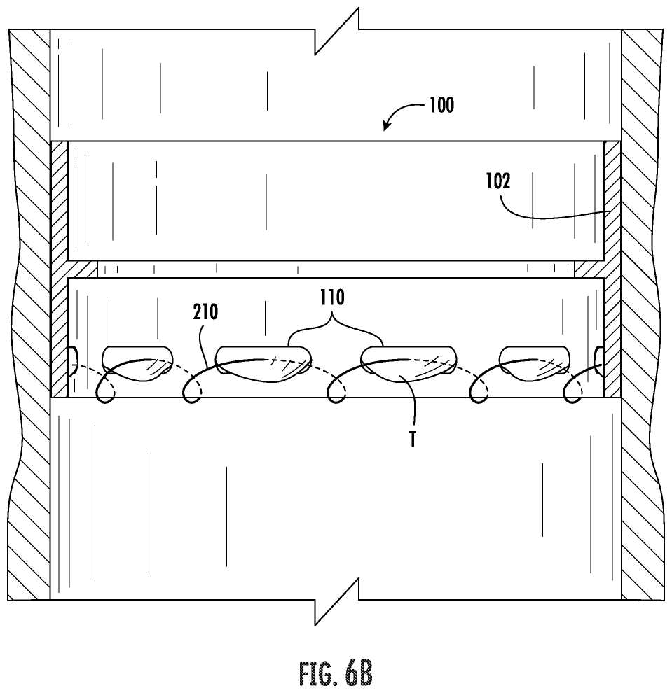

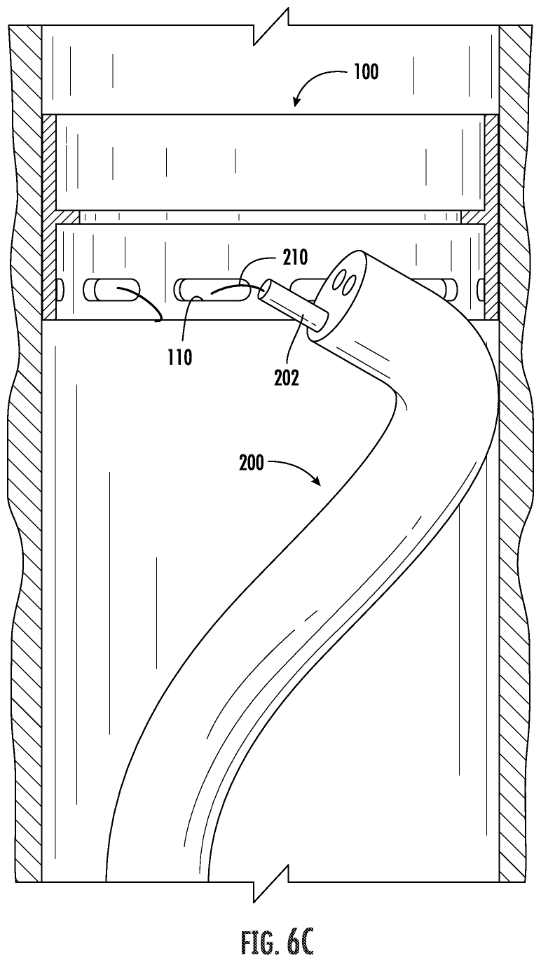

In the example of an embodiment illustrated in FIG. 6A, the working instrument 202 includes grasper jaws 204 configured to grasp tissue at the deployment site to facilitate engagement of the grasped tissue with the implantable medical device 100. For instance, the grasper jaws 204 may extend through tissue-engagement features 110 in the form of apertures through the wall 102 of the implantable medical device 100 to grasp tissue at the deployment site and to pull the tissue through the tissue-engagement features 110. Such steps may be sufficient to induce tissue ingrowth to secure the implantable medical device 100 in place at the deployment site. If desired, to enhance or to assure attachment of the tissue and the implantable medical device 100, a separate tissue connector 210 may be used, such as illustrated, to couple the tissue and the implantable medical device 100. The tissue connector 210 may be passed through the tissue and implantable medical device 100 utilizing a working instrument 202 with grasper jaws 204, such as illustrated in FIG. 6A, or with another working instrument 202 suitable for such task and known to those of ordinary skill in the art, or with any of a variety of internal suturing instruments known or heretofore known to those of ordinary skill in the art such as to provide a running stitch of suture (the present disclosure not being limited in this regard). The tissue connector 210 may be passed simply through the tissue extending through the tissue-engagement features 110 formed in the wall 102 of the implantable medical device 100, such as illustrated in FIG. 6A. Optionally, the tissue connector 210 is also looped around portions of the implantable medical device 100 as well, such as around portions of the wall 102 adjacent to the tissue-engagement features 110 and a free end of the implantable medical device 100, as illustrated in FIG. 6B. In some embodiments, however, tissue is not pulled through the tissue-engagement features 110, and the tissue connector 210 is passed through the tissue-engagement features 110 to be then passed through adjacent tissue, such as illustrated in FIG. 6C.

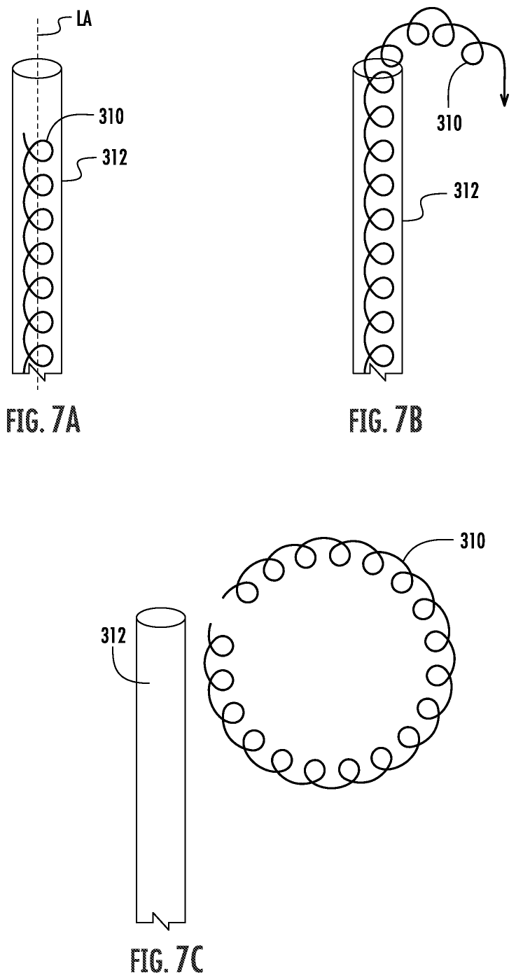

The tissue connector 210 may be any suitable, preferably biocompatible, element known to those of ordinary skill in the art for coupling tissue to another element (tissue or otherwise), such as a suture, a thread, a wire, a tether, a filament, a band, a staple, etc., typically at least somewhat elongated and flexible. For instance, in the example of an embodiment illustrated in FIG. 6A, the tissue connector 210 may be elongated and flexible, such as a suture. The tissue connector 210 may be sufficiently flexible to be looped, such as illustrated in FIG. 6B and FIG. 6C. In some embodiments, the tissue connector 210 may be a pre-coiled element 310 such as illustrated in FIGS. 7A-7C. More particularly, the pre-coiled element 310 illustrated in FIG. 7A has a pre-set coiled or helicoidal configuration and may be delivered within a flexible tubular element such as a delivery sheath 312. Upon exiting the delivery sheath 312, the pre-coiled element 310 maintains a coiled configuration about a longitudinal axis LA thereof, such as illustrated in FIG. 7B. Such configuration may facilitate engagement with and coupling of the tissue and the implantable medical device 100. Moreover, once the entire pre-coiled element 310 is deployed, the pre-coiled element 310 may also maintain a coiled configuration about a generally circular configuration, such as illustrated in FIG. 7C. Such generally circular configuration may be configured to complement the cross-sectional configuration of the implantable medical device 100, such as by having a common radius of curvature, and thus may further facilitate engagement with and coupling of the implantable medical device 100 with tissue at the target area. Further details of an example of a pre-coiled element 310 are provided in U.S. Patent Publication No. 2021/0128126, published May 6, 2021, the entire contents of which application is incorporated herein for all purposes.

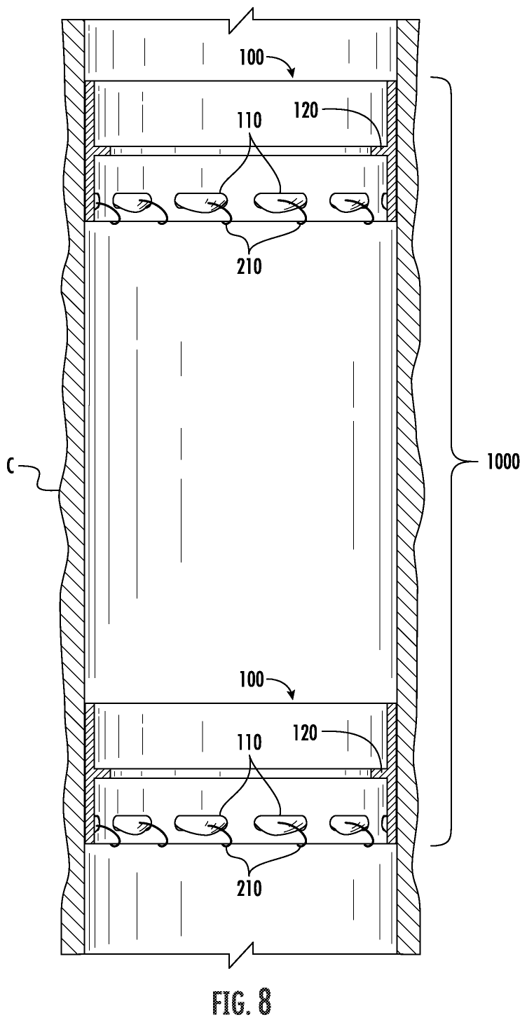

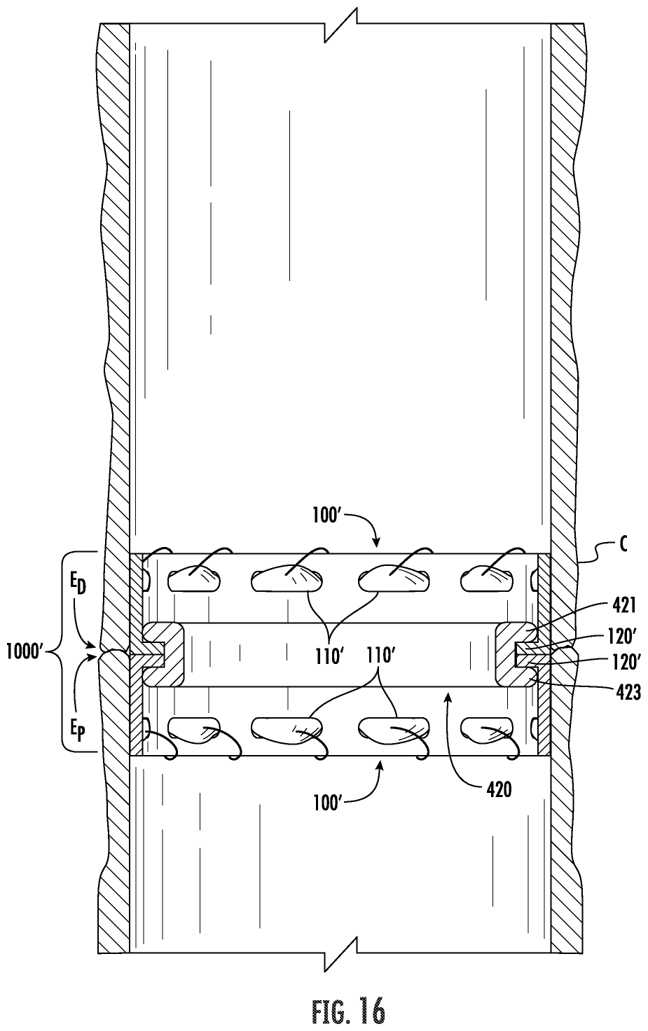

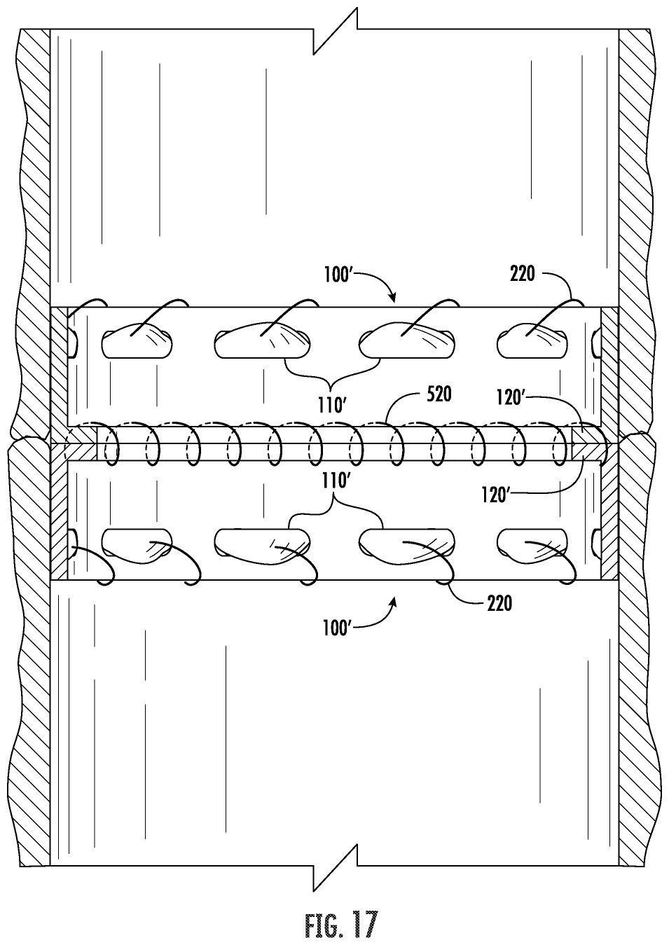

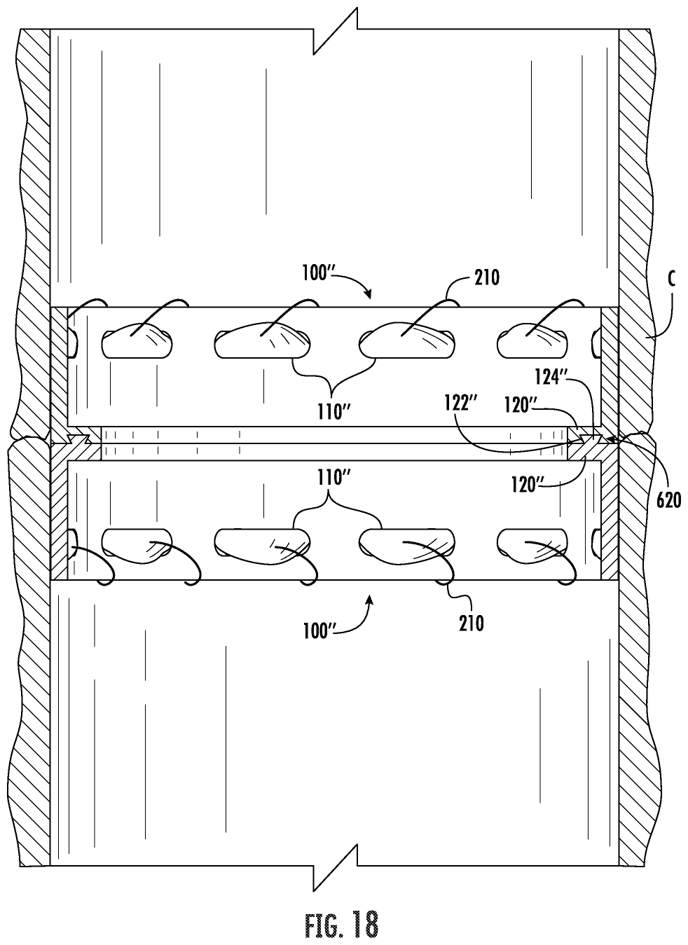

In the example of an embodiment illustrated in FIG. 8, both implantable medical devices 100 of an implantable medical device system 1000 formed and deployed in accordance with various principles of the present disclosure may be anchored with respect to tissue in a similar manner and general orientation. However, variations in implantable medical devices 100 and/or orientations thereof and/or manners of deployment and implantation thereof with respect to target tissue, such as with respect to each other, are within the scope and spirit of the present disclosure. For instance, although the implantable medical devices 100 illustrated in FIG. 8 are positioned within a colon C in the same orientation, the orientations may, instead be mirror images of each other. In other words, in some embodiments, the tissue-engagement features 110 of one implantable medical device 100 may face the support 120 of the other of the implantable medical device 100 of an implantable medical device system 1000, such as illustrated in FIG. 8. In other embodiments, the supports 120 of the respective implantable medical devices 100 may face each other, such as illustrated in FIG. 16, FIG. 17, and FIG. 18, as discussed in further detail below. Furthermore, it will be appreciated that although the implantable medical devices 100 of the implantable medical device system 1000 illustrated in FIG. 8 are similar or even substantially identical, the implantable medical devices 100 of an implantable medical device system 1000 formed in accordance with various principles of the present disclosure need not be identical or even similar (such as described in further detail below).

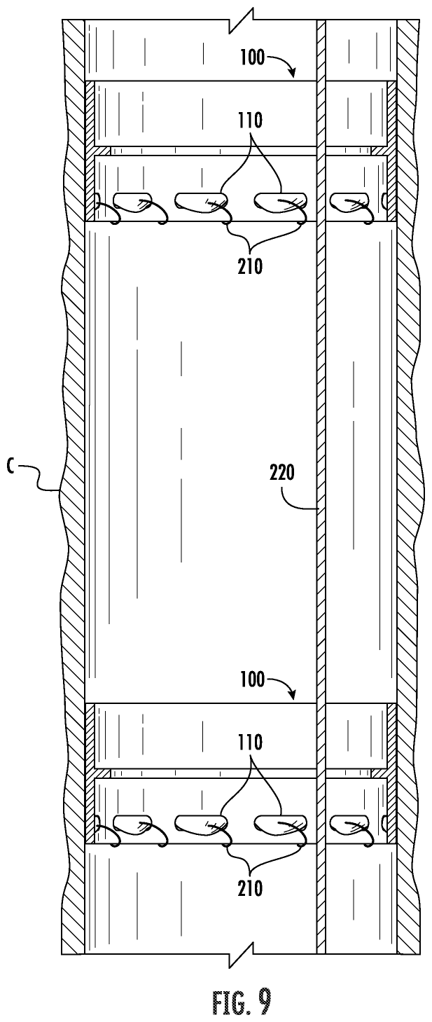

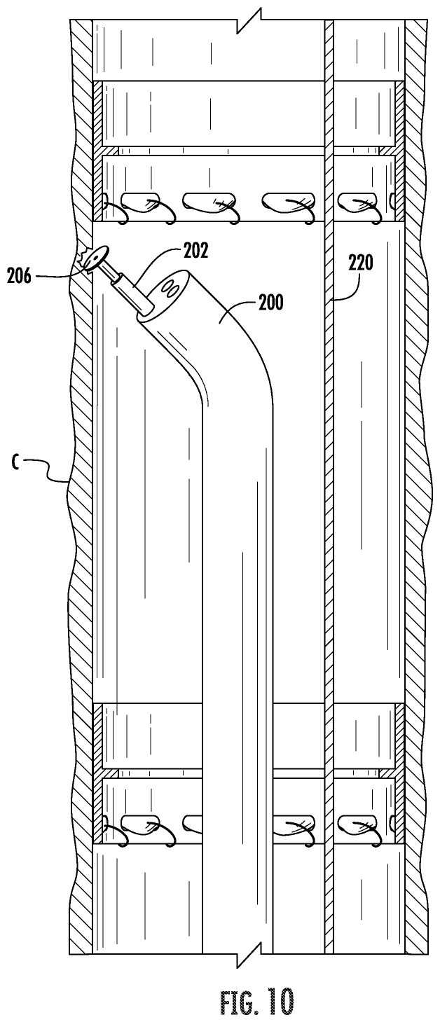

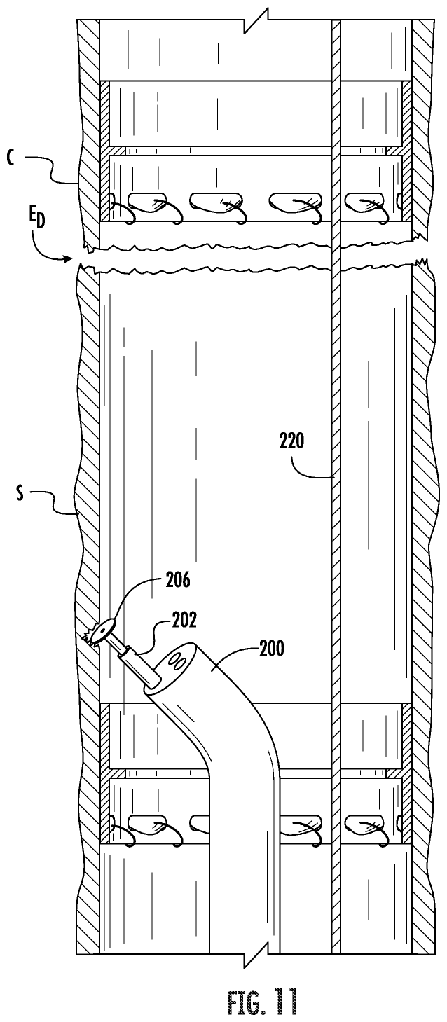

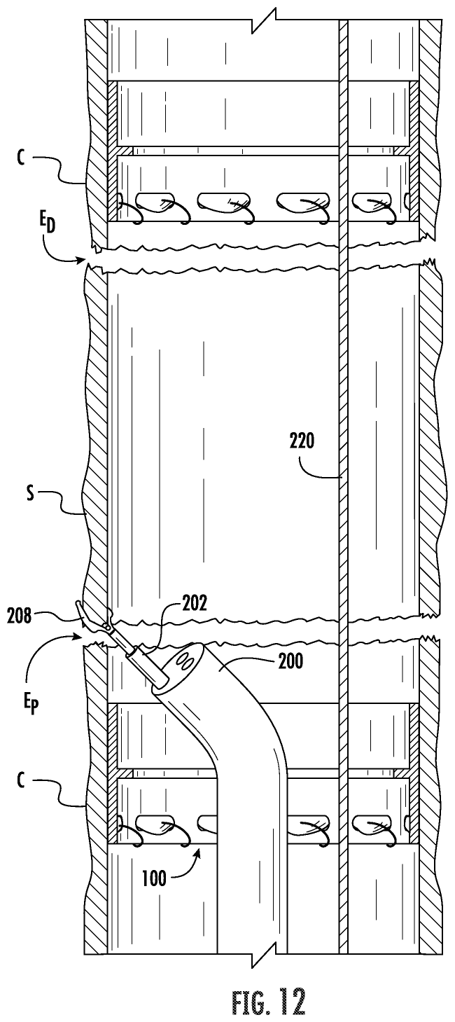

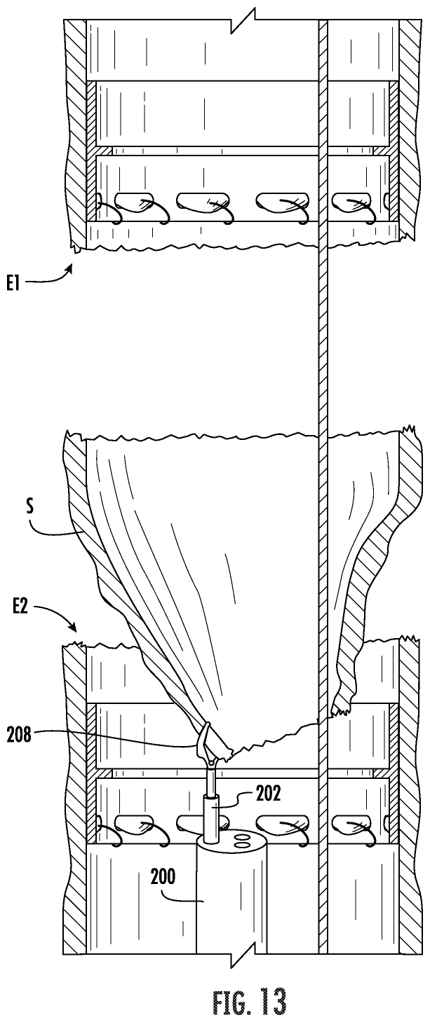

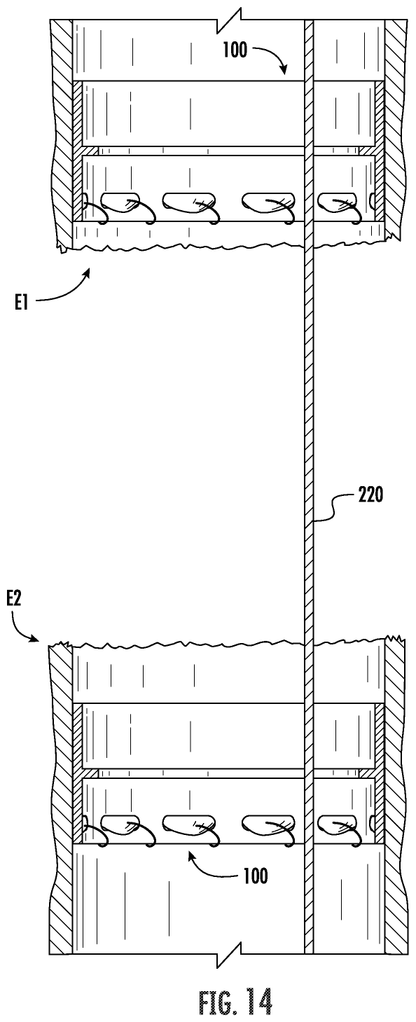

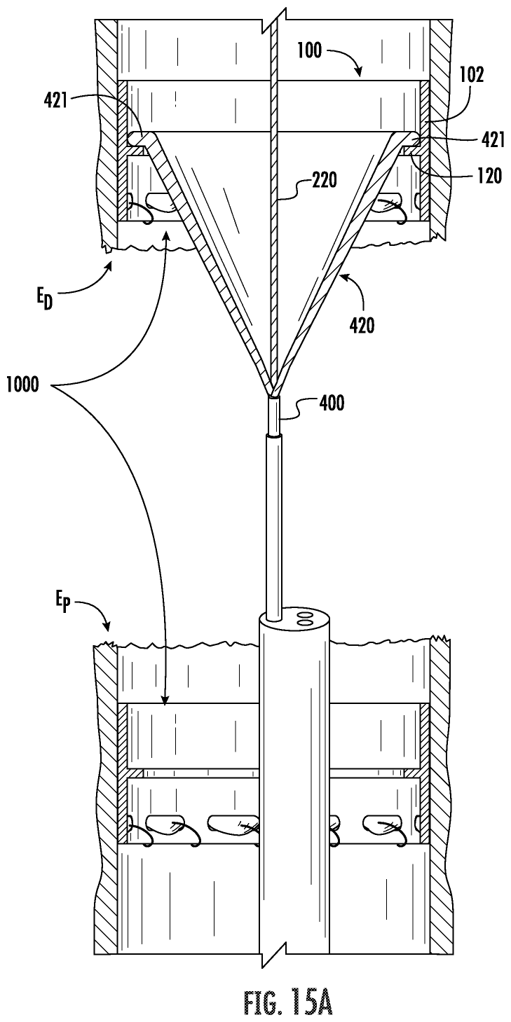

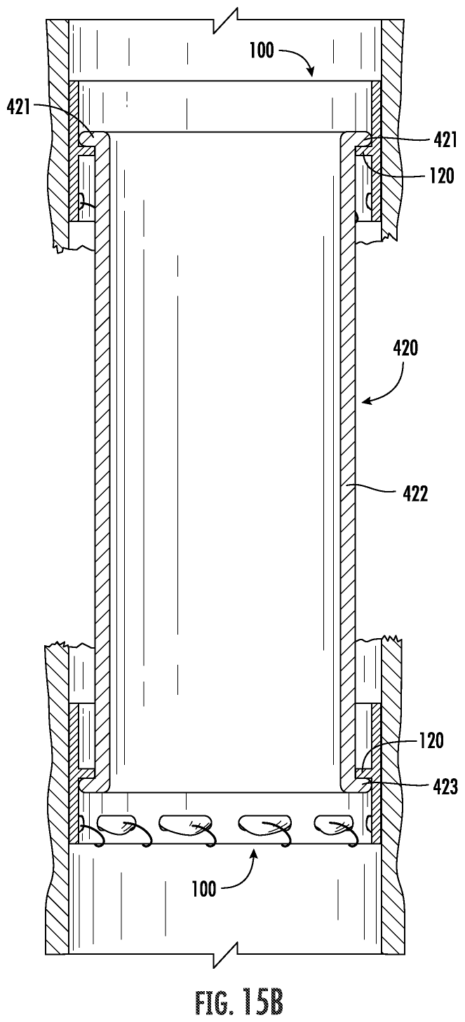

In an embodiment in which separate implantable medical devices 100 are deployed in a common body lumen, one of the implantable medical devices 100 may be deployed first, and then the second implantable medical device 100 may be deployed. For instance, a first distal implantable medical device 100 may be deployed, and then a second proximal implantable medical device 100 may be deployed. Each implantable medical device 100 may be anchored with respect to the tissue upon deployment or once both implantable medical devices 100 have been deployed. Once both implantable medical devices 100 have been deployed and implanted in a common body lumen, spaced apart from each other, such as illustrated in FIG. 8, the common lumen may be cut between the spaced-apart implantable medical devices 100. Before cutting the body lumen, it generally is desirable to connect the ends of the lumen on either side of the cut to be made so that the cut ends may be reconnected after resecting tissue therebetween. For instance, a core wire or guidewire with an anchoring balloon or other form of a tether/anchor element may be used to hold tissue distal to the cut with respect to tissue proximal to the cut. As illustrated in FIG. 9, a tether 220 may be inserted through the body lumens to maintain the positions and/or control of the portions of the body lumen (in the illustrated example, the colon C) in which the implantable medical devices 100 are implanted. Once a distal end of the tether 220 (or any other acceptable tether/anchor element known to those of ordinary skill in the art) is coupled to the distal implantable medical device 100 or to tissue distal to the section of the body lumen to be cut (e.g., with a knot or alpha-loop or otherwise, such as through the tissue-engagement features 110 in the implantable medical devices 100), a working instrument 202 including a cutting end 206 (a cutting tool known to those of ordinary skill in the art, such as a blade, electrosurgical knife, scalpel, etc.) may be delivered (such as through the endoscope 200) to cut the tissue between the implantable medical devices 100, such as illustrated in FIG. 10. Tissue proximal to the distal one of the implantable medical devices 100 may be cut to separate the portion of the colon C in which the distal implantable medical device 100 is anchored from the remaining (e.g., more proximal) portion of the colon C. If the colon C is fully resected, the distal implantable medical device 100 remains proximal to the distal cut end E_{D} of the colon C, such as illustrated in FIG. 11. The cutting end 206 may then be moved proximally to cut tissue distal to the proximal implantable medical device 100 and proximal to the first cut which was made, such as illustrated in FIG. 11. Once cutting of tissue proximal to the first cut has been completed, a section S of the colon C to be removed is defined, as well as a second cut end E_{P}, distal to the proximal implantable medical device 100, such as illustrated in FIG. 12. A working instrument 202 with grasper jaws 208 may be inserted to grasp the proximal end of the resected section S of the colon C, such as illustrated in FIG. 12. It will be appreciated that the working instrument 202 with grasper jaws 208 may be the same as the above-described working instrument 202 with grasper jaws 208, or a different instrument, the present disclosure not being limited in this manner. Once the grasper jaws 208 of the working instrument 202 have securely engaged the resected section S of tissue, the working instrument 202 may be proximally withdrawn, such as illustrated in FIG. 13, to remove the resected section S. As illustrated in FIG. 14, the tether 220 maintains the first implantable medical device 100 and the second implantable medical device 100 with their respective cut ends E_{D}, E_{P} spaced apart from but generally facing each other, as illustrated in FIG. 14. The implantable medical devices 100 may now be coupled together to join the cut ends E_{D}, E_{P} of the body lumen to form an anastomosis and to reestablish flow of material through the body lumen. The implantable medical devices 100 may be coupled together in a variety of manners and with a variety of connectors formed in accordance with various principles of the present disclosure, such as illustrated, for example, in FIG. 15A, FIG. 15B, FIG. 16, FIG. 17, and FIG. 18.

In the example of an embodiment illustrated in FIG. 15A and FIG. 15B, the implantable medical devices 100 are coupled together with a tubular connector 420. The tubular connector 420 may be in the form of a stent or scaffold such as any known to those of ordinary skill in the art (e.g., similar to a stent or scaffold as described above with reference to the structure of the implantable medical device 100). The tubular connector 420 may be delivered within a delivery sheath 400 to the deployment site of the implantable medical device system 1000, such as illustrated in FIG. 15A, in a generally compact configuration. The delivery sheath 400 may be guided over the tether 220 to the deployment site of the implantable medical device system 1000. A distal end 421 of the tubular connector 420 may be deployed first, distal to the distal cut end E_{D}, such as by proximal withdrawal of the sheath 400. In accordance with various principles of the present disclosure, the support 120 of the implantable medical device 100 adjacent the distal cut end E_{D} is configured to support the distal end 421 of the tubular connector 420, and the distal end 421 of the tubular connector 420 is configured to engage the support 120 of the implantable medical device 100 to secure the tubular connector 420 with respect to the implantable medical device 100. The delivery sheath 400 may then be retracted proximally to deliver the proximal end 423 to the implantable medical device 100 implanted at the proximal cut end E_{P} of the body lumen. The proximal end 423 of the tubular connector 420 is configured to engage the support 120 of the implantable medical device 100, and the support 120 of the implantable medical device 100 adjacent the proximal cut end E_{P} is configured to support 120 the proximal end 423 of the tubular connector 420 to secure the tubular connector 420 with respect to the implantable medical device 100, such as illustrated in FIG. 15B. In some embodiments, the proximal end 423 of the tubular connector 420 and/or the distal end 423 of the tubular connector 420 are formed as flanges or walls extending radially outwardly from the tubular wall 422 of the tubular connector 420. It will be appreciated by those of ordinary skill in the art that imaging (e.g., fluoroscopy), and/or precise measurement of the resected section S and the distance between the cut ends E_{D}, E_{P} of the body lumen, and/or other markers associated with the endoscope 200 may be used to determine the distance the delivery sheath 400 is to be proximally retracted in order to accurately deliver and deploy the proximal end 423 of the tubular connector 420 with respect to the implantable medical device 100 adjacent the proximal cut end E_{P}, the details of which are not critical to the present disclosure, and/or if the distal cut end E_{D}, should be drawn proximally closer to the proximal cut end E_{P}. It will be appreciated that the size, shape, configuration, dimensions, and/or resistance to flexure of the distal end 421 and proximal end 423 of the tubular connector 420, and/or the size, shape, configuration, and/or dimensions of the supports 120 of the respective implantable medical devices 100 may be selected to secure the engagement therebetween to hold the tubular connector 420 in place with respect to the cut ends E_{D}, E_{P} of the body lumen to form an anastomosis therebetween, in manners appreciated by those of ordinary skill in the art.

The length of the tubular connector 420 may be selected based on the distance between the cut ends E_{D}, E_{P} to span the distance between the cut ends E_{D}, E_{P} and to form a passage or lumen therebetween. As may be appreciated with reference to FIG. 15B, the tubular connector 420 forms a passage or lumen coupling the separated body lumens (e.g., as an extension or artificial anastomosis), and thus the walls thereof may be coated to prevent flow of fluids through the walls thereof (in a direction from the inner surface to the outer surface of the wall), rather than generally longitudinally through the lumen within the tubular connector 420 from the upstream body lumen to the downstream body lumen connected by the tubular connector 420. The coating may be any known or heretofore known biocompatible material which may prevent flow of fluids therethrough, including, without limitation, silicone, styrene isoprene butadiene (SIBS), polytetrafluoroethylene (PTFE), expanded polytetrafluoroethylene (ePTFE), ethylene tetrafluoroethylene (ETFE), fluorinated ethylene propylene (FEP), urethane, polyurethane, polyvinylidene chloride (PVC), polyether block amides (PEBA), polyimide, polyethylene, polyethylene terephthalate (PET), polysulfone, nylon, polytrimethylene terephthalate, polyvinylidene difluoride (PVDF), polyester, polyether-ester, polypropylene, polyolefin, polystyrene, polynapthalene, polyethylene napthalate (PEN), polyetherether ketone (PEEK), polyetherimide, polyphenylene sulfide (PPS), polyphenylene oxide (PPO), perfluoro(propyl vinyl ether) (PFA), polyparaphenylene teraphthalamide, polybutylene terephthalate (PBT), polyoxymethylene (POM), polyether block ester, poly(styrene-butadiene-styrene) (SBS), styrene-ethylene-butylene-styrene (SEBS), poly(styrene-b-isobutylene-b-styrene), ethylene vinyl alcohol, ethylene vinyl acetate copolymers (EVA), polycarbonates, ionomers, thermoplastic elastomers (TPE), epoxy, etc., including copolymers and/or combinations thereof.

It will be appreciated that in some embodiments, a tubular connector need not span across a gap between cut ends E_{D}, E_{P} of a body lumen. For instance, in the example of an embodiment illustrated in FIG. 16, a tubular connector 420 shorter than the tubular connector 420 illustrated in FIG. 15A and FIG. 15B is extended between the implantable medical device 100′ implanted distal to the cut end E_{D}, and the implantable medical device 100′ implanted proximal to the cut end E_{P} to bring the implantable medical device 100′ closer together. For example, the implantable medical devices 100′ may be brought into abutting contact with each other. It is noted that the implantable medical devices 100′ are arranged in a mirror image orientation rather than in a similar orientation as in the example of an embodiment illustrated in FIG. 15A and FIG. 15B. It is further noted that the configuration of the implantable medical devices 100′ illustrated in FIG. 16 is somewhat different from the configuration of the implantable medical device 100 in that the support 120′ is located closer to, if not immediately located at, the free ends of the implantable medical devices 100 facing each other. The distal end 421′ may engage the support 120′ of the distal implantable medical device 100′ and the proximal end 423′ may engage the support 120′ of the proximal implantable medical device 100′ with the supports 120′ of the implantable medical devices 100 in abutting contact with each other.

In some aspects of the present disclosure, the implantable medical devices 100 are coupled with a connector other than a stent such as illustrated in FIG. 15A, FIG. 15B, and FIG. 16. For instance, in the example of an embodiment illustrated in FIG. 17, the implantable medical devices 100′ are brought together into abutting contact (e.g., with the supports 120′ thereof in abutting contact), and then coupled together with a separate elongated connector 520 (e.g., tether, wire, suture, string, etc.). In such embodiment, the supports 120′ of the implantable medical devices 100′ may include structures such as apertures or protrusions facilitating engagement of the connector 520 with the support 120′ to couple the implantable medical devices 100′ together securely. Moreover, it will be appreciated by those of ordinary skill in the art, abutting contact and a secure engagement of the supports 120′ may be desirable if not important to maintain flow of materials through the anastomosis formed upon connecting the implantable medical devices 100′ without flowing through the walls of the anastomosis.

In some aspects of the present disclosure, the implantable medical devices 100 are coupled directly to each other without the need of a separate connector element. For instance, in the example of an embodiment illustrated in FIG. 18, the supports 120″ of the implantable medical devices 100″ are configured to interlock or otherwise to engage each other to hold the implantable medical devices 100″ in abutting contact, connected to form an anastomosis. It will be appreciated that terms such as interlock, interengage, interfit, mate, etc., including grammatical forms thereof, may be used interchangeably herein without intent to limit to describe configurations allowing engagement of the implantable medical device to couple the implantable medical devices together without a separate connector. In the illustrated example of an embodiment, the support 120″ of one implantable medical device 100″ includes a groove 122″, and the support 120″ of one implantable medical device 100″ includes a protrusion 124″ configured to engage, mate with, fit within, etc., the groove 122. It will be appreciated that the present disclosure is not to be limited to a particular form or configuration of supports 120″, and that other configurations which allow secure engagement of implantable medical devices formed in accordance with various principles of the present disclosure and known to those of ordinary skill in the art are within the scope of spirit of the present disclosure.

It will be appreciated that reference to a body passage includes naturally-existing passages (e.g., the colon) as well as medically-created passages (e.g., a passage created with the use of a medical instrument, and not existing without medical intervention) or otherwise.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable medical device system configured to form an anastomosis across cut ends of a body lumen, said system comprising:
a first tubular implantable medical device;
a second tubular implantable medical device; and
a connector configured to couple said first tubular implantable medical device and said second tubular implantable medical device together;
wherein said first tubular implantable medical device and said second tubular implantable medical device include:
tissue-engagement features configured to facilitate coupling of said first and second tubular implantable medical devices with respect to a body lumen; and
a support configured to facilitate coupling of said first and second tubular implantable devices together.

2. The system of claim 1, wherein:
said tissue-engagement features are provided at a tissue-engaging region of each of said first and second implantable medical devices; and
said support includes a support at a device-engaging region of each of said first and second implantable medical devices.

3. The system of claim 1, wherein said tissue-engagement features comprise apertures.

4. The system of claim 1, wherein said tissue-engagement features are configured to allow at least one of a tissue connector or tissue to extend therethrough to anchor said first and second implantable medical devices with respect to the body lumen.

5. The system of claim 1, wherein said connector is a tubular connector formed separately from said first and second implantable medical devices, and has a first end engageable with the support of said first implantable medical device and a second end engageable with the support of said second implantable medical device to couple said first and second implantable medical devices together.

6. The system of claim 5, wherein said tubular connector couples said first and second implantable medical devices together with supports of said first and second implantable medical device spaced apart from each other.

7. The system of claim 6, wherein said tubular connector is coated to prevent passage of materials through the wall thereof.

8. The system of claim 5, wherein said tubular connector couples said first and second implantable medical devices together with supports of said first and second implantable medical device abuttingly engaging each other.

9. The system of claim 1, wherein said connector is an elongated tether-like connector formed separately from said first and second implantable medical devices and configured to be secured to said supports of said first and second implantable medical devices to couple said first and second implantable medical devices together in abutting engagement.

10. The system of claim 1, wherein said connector comprises interengaging features on said supports of said first and second implantable medical device configured to interengage each other to couple said first and second implantable medical devices together.

11. The system of claim 1, further comprising a tether coupling said first and second implantable medical devices together after the body lumen to which said first and second implantable medical devices are coupled has been cut between said first and second implantable medical devices.

12. An implantable medical device system comprising:
a first implantable medical device;
a second implantable medical device; and
a connector configured to couple said first implantable medical device and said second implantable medical device together;
wherein:
said first implantable medical device and said second implantable medical device each include:
tissue-engagement features configured to facilitate coupling of said first and second implantable medical devices with tissue; and
a support configured to facilitate engagement of said first and second implantable devices to couple said first and second implantable medical devices together; and
said connector establishes an anastomosis through said first implantable medical device and said second implantable medical device preventing flow of materials in a direction through walls of said first and second implantable medical devices.

13. The system of claim 12, wherein said tissue-engagement features comprise apertures configured to allow at least one of a tissue connector or tissue to extend therethrough.

14. The system of claim 12, wherein said connector is a tubular connector formed separately from said first and second implantable medical devices, and has a first end engageable with the support of said first implantable medical device and a second end engageable with the support of said second implantable medical device to couple said first and second implantable medical devices together.

15. The system of claim 14, wherein said tubular connector is a stent having a first end engageable with said support of said first implantable medical device and a second end engageable with said support of said second implantable medical device.

16. A method of forming an anastomosis after a colostomy, said method comprising:
implanting a first implantable medical device within a colon at a location distal to target tissue to be excised;
implanting a second implantable medical device within a colon at location proximal to target tissue to be excised;
excising the target tissue; and
joining device-engaging regions of the first and second implantable medical devices with a connector.

17. The method of claim 16, wherein implanting a first implantable medical device and implanting a second implantable medical device comprise extending at least one of tissue of the colon or a tissue connector through tissue-engagement features in each of the first implantable medical device and the second implantable medical device to anchor the first and second implantable medical devices with respect to the colon.

18. The method of claim 16, wherein joining device-engaging regions of the first and second implantable medical devices comprises extending a tubular stent between the first and second implantable medical devices to form an anastomosis therebetween.

19. The method of claim 16, wherein joining device-engaging regions of the first and second implantable medical devices comprises bringing the first and second devices into abutting engagement and coupling the first and second implantable medical devices together in abutting engagement to form an anastomosis through the first and second implantable medical devices.

20. The method of claim 16, further comprising coupling a portion of the colon distal to the first implantable medical device with a portion of the colon proximal to the second implantable medical device with a tether before joining the first and second implantable medical devices together.
